(19) United States
(12) Patent Application Publication
(10) Pub. No.: US 2026/0234196 A1
(43) Pub. Date: Aug. 13, 2026

(54) POLYPEPTIDE AND POLYPEPTIDE COMPOSITION SPECIFICALLY BINDING TO SARS-COV-2 SPIKE PROTEIN, AND PREPARATION METHOD AND USE THEREOF

(71) Applicants: INSTITUTE OF BASIC MEDICAL SCIENCES CHINESE ACADEMY OF MEDICAL SCIENCES, Beijing (CN); NATIONAL CENTER FOR NANOSCIENCE AND TECHNOLOGY, Beijing (CN)

(72) Inventors: Haiyan XU, Beijing (CN); Chen WANG, Beijing (CN); Tao WANG, Beijing (CN); Yanlian YANG, Beijing (CN); Jian LIU, Beijing (CN); Xiaocui FANG, Beijing (CN); Tao WEN, Beijing (CN); Jie MENG, Beijing (CN)

(21) Appl. No.: 18/279,992

(22) PCT Filed: Mar. 1, 2022
(86) PCT No.: PCT/CN2022/078627

§ 371 Date: Sep. 1, 2023

(30) Foreign Application Priority Data
Mar. 1, 2021 (CN) 2021102242518
Mar. 1, 2021 (CN) 2021102242522
Mar. 1, 2021 (CN) 2021102248001
Mar. 1, 2021 (CN) 2021102248228

(51) Int. Cl. C07K 14/00; A61K 38/00; A61P 31/14

(52) U.S. Cl. CPC C07K 14/001; A61P 31/14; A61K 38/00

(57) ABSTRACT

Disclosed in the present invention are a polypeptide and a polypeptide composition specifically binding to a SARS-CoV-2 spike protein, and preparation methods and the uses thereof. By means of binding to S1 protein, the polypeptide and the polypeptide composition can block the binding of SARS-CoV-2 virus to a key target protein angiotensin-converting enzyme 2 (ACE2) protein on the surface of a host cell thereof, thereby providing a feasible targeting drug for the treatment of Coronavirus disease 2019.

### TECHNICAL FIELD

The invention relates to the technical field of biomedicine, in particular to a polypeptide and a polypeptide composition that specifically binds to SARS-CoV-2 spike protein (especially SARS-CoV-2 surface spike S1 protein), and preparation method and use of the polypeptide and the polypeptide composition.

### INCORPORATION BY REFERENCE OF SEQUENCE LISTING

The sequence listing in ASCII text format, named as 42478_Substitute SequenceListing.txt of 4,096 bytes, created on Jun. 6, 2025, and submitted to the United States Patent and Trademark Office via Patent Center, is incorporated herein by reference.

### BACKGROUND

The pathogen of Coronavirus disease 2019 (referred to as COVID-19) is a new type of coronavirus SARS-CoV-2. The current treatment drugs for Coronavirus disease 2019 are mainly broad-spectrum antiviral drugs and drugs for relieving symptoms, and there is still a lack of effective targeted drugs.

### SUMMARY OF THE INVENTION

The main purpose of the invention, in the first aspect, is to provide a polypeptide composition specifically binding to SARS-CoV-2 spike protein, comprising one or more of polypeptide KVp-N, polypeptide KVp-R and polypeptide KVp-C. The polypeptide KVp-N contains the amino acid sequence shown in SEQ ID NO:1, the polypeptide KVp-R contains the amino acid sequence shown in SEQ ID NO:3, and the polypeptide KVp-C contains the amino acid sequence shown in SEQ ID NO:5.

The ends of the polypeptide KVp-N, polypeptide KVp-R and polypeptide KVp-C are modified with a sulfhydryl-containing peptide fragment; preferably, the amino acid sequence of the sulfhydryl-containing peptide fragment is CCPPPP (SEQ ID NO:7).

The amino acid sequence of the polypeptide KVp-N modified with the sulfhydryl-containing peptide fragment is shown in SEQ ID NO:2;

The amino acid sequence of the polypeptide KVp-R modified with the sulfhydryl-containing peptide fragment is shown in SEQ ID NO:4;

The amino acid sequence of the polypeptide KVp-C modified with the sulfhydryl-containing peptide fragment is shown in SEQ ID NO:6.

The polypeptide composition comprises at least one polypeptide listed above, and when comprising two or more polypeptides, the polypeptides are mixed in any proportion. Preferably, the three types of polypeptides of polypeptide KVp-N, polypeptide KVp-R and polypeptide KVp-C are mixed in an equimolar ratio.

In a second aspect, the invention provides a polypeptide that specifically binds to SARS-CoV-2 spike protein, named polypeptide KVp-N, which contains the amino acid sequence shown in SEQ ID NO:1.

In a third aspect, the invention provides a polypeptide that specifically binds to SARS-CoV-2 spike protein, named polypeptide KVp-R, which contains the amino acid sequence shown in SEQ ID NO:3.

In a fourth aspect, the invention provides a polypeptide that specifically binds to SARS-CoV-2 spike protein, named polypeptide KVp-C, which contains the amino acid sequence shown in SEQ ID NO:5.

The ends of the polypeptides are modified with a sulfhydryl-containing peptide fragment; preferably, the amino acid sequence of the sulfhydryl-containing peptide fragment is CCPPPP (SEQ ID NO:7).

The amino acid sequence of the polypeptide KVp-N is shown in SEQ ID NO:1 or SEQ ID NO:2; The amino acid sequence of the polypeptide KVp-R is shown in SEQ ID NO:3 or SEQ ID NO:4;

The amino acid sequence of the polypeptide KVp-C is shown in SEQ ID NO:5 or u.

In a fifth aspect, the invention provides a method for preparing the above-mentioned polypeptides, which are synthesized using a standard solid phase polypeptide synthesis method.

In a sixth aspect, the invention provides a method for preparing the above-mentioned polypeptide composition. After the polypeptides are synthesized using the standard solid phase polypeptide synthesis method, the polypeptide composition are obtained by mixing the polypeptides.

In a seventh aspect, the invention provides an use of the polypeptide or polypeptide composition above in the preparation of a medicine for treating Coronavirus disease 2019, preferably, the coronavirus is SARS-CoV-2.

In a eighth aspect, the invention provides an use of the polypeptide or polypeptide composition above in the preparation of an blocking agent for blocking the binding of SARS-CoV-2 surface spike S1 protein to angiotensin-converting enzyme 2 (ACE2); preferably, the angiotensin-converting enzyme 2 (ACE2) exists on the surface of ciliated bronchial epithelial cells or type II lung cells.

In the use, the polypeptide or polypeptide composition is used to specifically block the binding of the SARS-CoV-2 surface spike S1 protein to the angiotensin-converting enzyme 2 (ACE2). Specifically:

The polypeptide or polypeptide composition is used to specifically bind to the SARS-CoV-2 surface spike S1 protein to block the binding of the SARS-CoV-2 surface spike S1 protein to the angiotensin-converting enzyme 2 (ACE2); and/or

The polypeptide or polypeptide composition is used to specifically bind to the RBD domain of the SARS-CoV-2 surface spike S1 protein subunit to block the binding of the SARS-CoV-2 surface spike S1 protein to the angiotensin-converting enzyme 2 (ACE2).

Based on the above aspects, it has been proved by experiments that the polypeptide or polypeptide composition provided by the invention that specifically binds to the SARS-CoV-2 surface spike S1 protein can block the binding of SARS-CoV-2 virus to the key target protein angiotensin-converting enzyme 2 (ACE2) protein on the surface of its host cell by binding to the S1 protein, thereby potentially providing a feasible targeting drug for the treatment of Coronavirus disease 2019.

### BRIEF DESCRIPTION OF THE DRAWINGS

FIGS. 1A-1C depict the detection curves of the binding ability of polypeptide KVp-N1, polypeptide KVp-R14, and polypeptide KVp-C15 to S1 protein, respectively;

FIGS. 2A-1C depict the detection curves of the binding ability of polypeptide KVp-N1, polypeptide KVp-R14, and polypeptide KVp-C15 to RBD protein, respectively;

FIGS. 3A-3C depict the Western Blot detection gels figures of the ability of S1 protein binding to human lung cancer cell line A549 cells after co-incubating with different concentrations of polypeptide KVp-N1, polypeptide KVp-R14, and polypeptide KVp-C15, respectively;

FIG. 3D depicts the Western Blot detection gels figure of the ability of S1 protein binding to human lung cancer cell line A549 cells after co-incubating with polypeptide KVp-N1, polypeptide KVp-R14, polypeptide KVp-C15 and the polypeptide composition, respectively;

FIGS. 4A-4C depict the Western Blot detection gels figures of the ability of RBD protein binding to A549 cells after co-incubating with different concentrations of polypeptide KVp-N1, polypeptide KVp-R14, and polypeptide KVp-C15, respectively;

FIGS. 5A-5C depict the Western Blot detection gels figures of the ability of S1 protein binding to 293T-ACE2⁺ cells after co-incubating with different concentrations of polypeptide KVp-N1, polypeptide KVp-R14, and polypeptide KVp-C15, respectively;

FIGS. 6A-6C depict the Western Blot detection gels figures of the ability of RBD protein binding to 293T-ACE2⁺ cells after co-incubating with different concentrations of polypeptide KVp-N1, polypeptide KVp-R14, and polypeptide KVp-C15, respectively;

FIG. 6D depicts the Western Blot detection gels figure of the ability of RBD protein binding to 293T-ACE2⁺ cells after co-incubating with polypeptide KVp-N1, polypeptide KVp-R14, polypeptide KVp-C15 and the polypeptide composition, respectively;

FIG. 7 is the histograms of the infection rate statistical results that SARS-CoV-2 pseudoviruses infect 293T-ACE2⁺ cells after co-incubating with the polypeptides; wherein panel A shows co-incubating with polypeptide KVp-N1 or polypeptide KVp-N1N, panel B shows co-incubating with polypeptide KVp-R14 or polypeptide KVp-R14N, and panel C shows co-incubating with polypeptide KVp-C15 or polypeptide KVp-C15N;

FIG. 7D is the histogram of the infection rate statistical results that SARS-CoV-2 pseudoviruses infect 293T-ACE2⁺ cells after co-incubating with a single polypeptide and the polypeptide composition, respectively;

FIGS. 8A-8C are the histograms of the infection rate statistical results that SARS-CoV-2 pseudoviruses infect 293T-ACE2⁺ cells after co-incubating with different concentrations of polypeptide KVp-N1N, polypeptide KVp-R14N, and polypeptide KVp-C15N, respectively;

FIGS. 9A-9C are the histograms of the statistical results that different concentrations of polypeptide KVp-N1, polypeptide KVp-R14, and polypeptide KVp-C15 effects the activity of 293T-ACE2⁺ cells, respectively;

FIGS. 10A-10C are the histograms of the statistical results that different concentrations of polypeptide KVp-N1N, polypeptide KVp-R14N, and polypeptide KVp-C15N effects the activity of 293T-ACE2⁺ cells, respectively.

### DETAILED DESCRIPTION

Studies have confirmed that SARS-CoV-2 can bind to angiotensin-converting enzyme 2 (ACE2) on the surface of ciliated bronchial epithelial cells and type II lung cells through the RBD domain of its surface spike protein S1 subunit, causing cell infection, thereby enabling people suffering from Coronavirus disease 2019.

A plurality of polypeptides that specifically bind to the SARS-CoV-2 surface spike S1 subunit or its RBD domain have been designed in the invention. The polypeptides are used to block the binding of SARS-CoV-2 to the key target protein angiotensin-converting enzyme 2 (ACE2) protein on the surface of its host cell. The polypeptides are respectively named polypeptide KVp-N, polypeptide KVp-R, and polypeptide KVp-C. These polypeptides are all prepared by artificial synthesis.

Wherein, the polypeptide KVp-N contains the amino acid sequence GDGVYYPRDVFDSSVLDSTQR (SEQ ID NO:1) (denoted as KVp-N1N); or the end of KVp-N1N is modified with a peptide fragment CCPPPP (SEQ ID NO:7), and the amino acid sequence of the modified polypeptide KVp-N may be CCPPPPGDGVYYPRDVFDSSVLDSTQR (SEQ ID NO:2) (denoted as KVp-N1). The purpose of introducing a sulfhydryl group (introduced by the peptide fragment CCPPPP (SEQ ID NO:7)) into KVp-N1 is to enable the polypeptide to bind to gold-containing substances (such as gold nanoparticles) through the sulfhydryl group, which will help to expand the use performance of the polypeptide, such as detecting and identifying the polypeptide in its use.

The polypeptide KVp-R contains the amino acid sequence GDLFDDSNLDPFRDRISTRR (SEQ ID NO:3) (denoted as KVp-R14N); or the end of KVp-R14N is modified with a peptide fragment CCPPPP (SEQ ID NO:7), and the amino acid sequence of the modified polypeptide KVp-R may be CCPPPPGDLFDDSNLDPFRDRISTRR (SEQ ID NO:4) (denoted as KVp-R14). The purpose of introducing a sulfhydryl group (introduced by the peptide fragment CCPPPP (SEQ ID NO:7)) into KVp-R14 is to enable the polypeptide to bind to gold-containing substances (such as gold nanoparticles) through the sulfhydryl group, which will help to expand the use performance of the polypeptide, such as detecting and identifying the polypeptide in its use.

The polypeptide KVp-C contains the amino acid sequence GDGVYYPRDVFDSSVLDSTQR (SEQ ID NO:5) (denoted as KVp-C15N); or the end of KVp-C15N is modified with a peptide fragment CCPPPP (SEQ ID NO:7), and the amino acid sequence of the modified polypeptide KVp-C may be CCPPPPGDGVYYPRDVFDSSVLDSTQR (SEQ ID NO:6) (denoted as KVp-C15). The purpose of introducing a sulfhydryl group (introduced by the peptide fragment CCPPPP (SEQ ID NO:7)) into KVp-C15 is to enable the polypeptide to bind to gold-containing substances (such as gold nanoparticles) through the sulfhydryl group, which will help to expand the use performance of the polypeptide, such as detecting and identifying the polypeptide in its use.

Based on these polypeptides, the invention also provides a polypeptide composition, which comprises at least one polypeptide that can specifically bind to SARS-CoV-2 surface spike protein S1 subunit.

The invention proves through experiments that the above-mentioned polypeptides and composition thereof can block the binding of S1 protein and ACE2 protein, thereby preventing SARS-CoV-2 from infecting epithelial cells and lung cells, so that these polypeptides and composition thereof can be used as a medicine for targeted treatment of Coronavirus disease 2019.

The embodiments of the invention will be described in detail below in combination with examples. Where the specific technology or conditions are not specified in the examples, they shall be carried out in accordance with the technology or conditions described in the literature in the field or in accordance with the product specification. The reagents or instruments used that do not indicate the manufacturer are conventional products that can be purchased through proper channels. The sources of biological materials used in the examples are extensive, and any biological materials that can be obtained without violating laws and ethics can be replaced according to the tips in the examples. Unless otherwise specified, the materials or reagents with the same name in each examples have the same content.

The phosphate buffer used in the following examples is 1×PBS solution (pH=7.4), unless otherwise specified.

### Example 1: Polypeptide, Polypeptide Composition and Preparation Thereof

In this example, a plurality of polypeptides targeting SARS-CoV-2 surface spike protein S1 were prepared by standard solid phase polypeptide synthesis, code-named KVp-N1N and KVp-N1 (collectively referred to as polypeptide KVp-N), KVp-R14N and KVp-R14 (collectively referred to as polypeptide KVp-R), KVp-C15N and KVp-C15 (collectively referred to as polypeptide KVp-C), and the amino acid sequences of these polypeptides are shown in Table 1 below.

TABLE 1
Amino acid sequence of polypeptide

| Name | Sequence |
|---|---|
| KVp-N1N | GDGVYYPRDVFDSSVLDSTQR |
| | (SEQ ID NO: 1) |
| KVp-N1 | CCPPPPGDGVYYPRDVFDSSVLDSTQR |
| | (SEQ ID NO: 2) |
| KVp-R14N | GDLFDDSNLDPFRDRISTRR |
| | (SEQ ID NO: 3) |
| KVp-R14 | CCPPPPGDLFDDSNLDPFRDRISTRR |
| | (SEQ ID NO: 4) |
| KVp-C15N | GDGVYYPRDVFDSSVLDSTQR |
| | (SEQ ID NO: 5) |
| KVp-C15 | CCPPPPGDGVYYPRDVFDSSVLDSTQR |
| | (SEQ ID NO: 6) |

The polypeptides in Table 1 were identified as target polypeptides by HPLC and MS. The six polypeptides were all white powder with purity≥98%. Among them, the polypeptides code-named KVp-N1, KVp-R14 and KVp-C15 are modified by introducing CCPPPP (SEQ ID NO:7) at the ends of the polypeptides code-named KVp-N1N, KVp-R14N and KVp-C15N respectively. A sulfhydryl group is introduced in the polypeptide KVp-N1, KVp-R14 and KVp-C15 by this modification, which can enable these polypeptides to bind to gold (such as gold nanoparticles) (for example, this binding is used to detect the binding ability of the polypeptide in Example 2).

The polypeptide composition in this example is composed of at least two polypeptides in Table 1. For example, it may be a mixture of KVp-N1N and KVp-R14, or a mixture of KVp-N1, KVp-C15 and KVp-C15N, or a mixture of KVp-N1, KVp-R14 and KVp-C15, or a mixture of KVp-R14, KVp-N1N, KVp-R14N and KVp-C15, or a mixture of KVp-N1, KVp-R14, KVp-C15, KVp-N1N and KVp-C15N, or a mixture of KVp-N1, KVp-R14, KVp-C15, KVp-N1N, KVp-R14N and KVp-C15N; and the mixing ratio is not limited.

In the following examples, in order to verify the biological effects of the polypeptides and their compositions, each polypeptide was prepared into a stock solution with a concentration of 10 mM with complete medium (commercially available), respectively; and the stock solutions of each polypeptide were mixed in a certain proportion to form a stock solution of the polypeptide composition. In order to detect the binding ability of the polypeptide composition, each polypeptide was prepared into a polypeptide solution with a concentration of 0.5 mg/mL with double distilled water (ddH₂O), respectively; and the polypeptide solutions were mixed in a certain proportion to form a polypeptide composition solution.

### Example 2: The Binding Ability of Polypeptide

In this example, the binding ability of the polypeptide KVp-N1, KVp-R14, and KVp-C15 (the sulfhydryl groups in these polypeptides are used in the detection) synthesized in Example 1 to SARS-CoV-2 S1 protein or its RBD segment (both purchased from Beijing Sino Biological Science and Technology Co., Ltd.) were detected by using the quartz crystal microbalance method (QCM) respectively; where the Qsense® four-channel dissipative quartz microbalance used was purchased from Biolin Scientific.

### Experiment 2.1: The Binding Ability of the Polypeptides to S1 Protein

(1) Clean the gold-coated quartz chip of the Qsense® four-channel dissipative quartz microbalance three times with cleaning solution (volume ratio of concentrated sulfuric acid and hydrogen peroxide is 3:1) for three minutes each time, and rinse with ddH₂O;
(2) According to the operating instructions of Qsense® four-channel dissipative quartz microbalance, the polypeptide solutions with a concentration of 0.5 mg/mL of KVp-N1, KVp-R14, KVp-C15 and the gold-coated quartz chips were incubated respectively at room temperature for 30 minutes, allowing the polypeptide KVp-N1, KVp-R14, and KVp-C15 molecules to bind to the gold-coated quartz chips through sulfhydryl groups;
(3) Rinse the chips with ddH₂O and dry it with nitrogen. The chips were embedded in the reaction chamber, and the S1 protein with a concentration of 13 nM prepared in PBS was used as the mobile phase, and flowed through the gold-coated quartz chips through pipelines, and the binding of S1 protein to the polypeptides KVp-N1, KVp-R14, and KVp-C15 was detected by QCM, respectively.

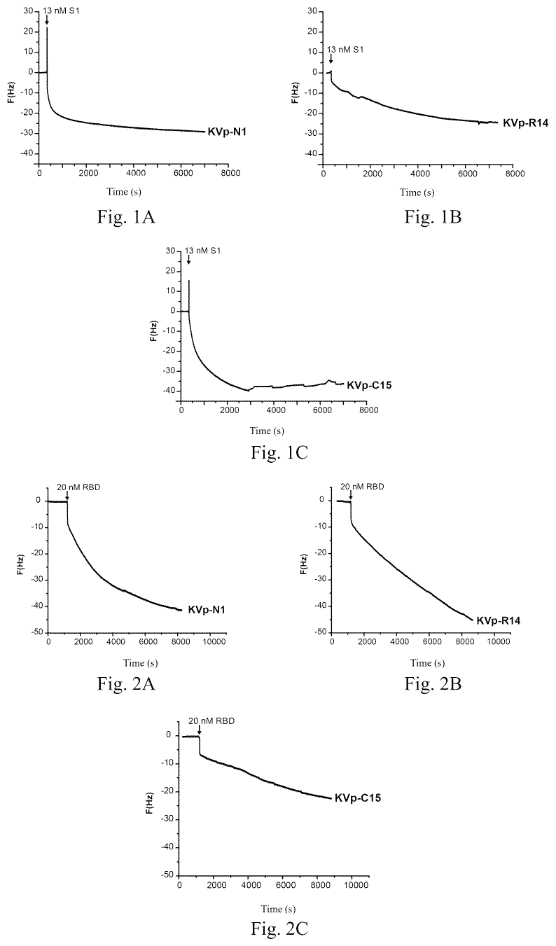

The results are shown in FIGS. 1A-1C, wherein FIG. 1A shows the binding curve of S1 protein to the polypeptide KVp-N1 detected by QCM, FIG. 1B shows the binding curve of S1 protein to the polypeptide KVp-R14 detected by QCM, and FIG. 1C shows the binding curve of S1 protein to the polypeptide KVp-C15 detected by QCM. It can be seen that when the S1 protein mobile phase flows through the pipelines, the oscillation frequency of the gold-coated quartz chips decreases, indicating that S1 protein is bound to the polypeptide KVp-N1, KVp-R14, and KVp-C15, respectively, that is, the polypeptides KVp-N1, KVp-R14, and KVp-C15 all can bind to S1 protein of SARS-CoV-2.

### Experiment 2.2: The Binding Ability of the Polypeptides KVp-N1, KVp-R14, and KVp-C15 to RBD Segment Protein

The specific operation of Experiment 2.2 was the same as that in Experiment 2.1 above, except that the RBD protein solution prepared in PBS with a concentration of 20 nM was used to replace the S1 protein solution as the mobile phase.

The QCM detection results of the binding ability of the polypeptides KVp-N1, KVp-R14, and KVp-C15 to RBD segment are shown in FIGS. 2A-2C, respectively, wherein FIG. 2A shows the binding curve of RBD segment to the polypeptide KVp-N1 detected by QCM, FIG. 2B shows the binding curve of RBD segment to the polypeptide KVp-R14 detected by QCM, and FIG. 2C shows the binding curve of RBD segment to the polypeptide KVp-C15 detected by QCM. It can be seen that when the RBD protein mobile phase flows through the pipelines, the oscillation frequency of the gold-coated quartz chips decreases, indicating that RBD protein is bound to the polypeptides KVp-N1, KVp-R14, and KVp-C15, respectively, that is, the polypeptides KVp-N1, KVp-R14, and KVp-C15 all can bind to RBD segment of SARS-CoV-2.

The above experimental results prove that the polypeptide KVp-N (KVp-N1N and KVp-N1), the polypeptide KVp-R (KVp-R14N and KVp-R14), and the polypeptide KVp-C (KVp-C15N and KVp-C15) can bind to S1 protein of SARS-CoV-2 or RBD segment thereof.

### Example 3: Polypeptide and Composition Thereof Prevent the Binding of S1 Protein and RBD Protein to Target Cells

In this example, the polypeptides KVp-N1, KVp-R14, KVp-C15 synthesized in Example 1 and their equimolar concentration mixture (i.e. the polypeptide composition composed of equimolar concentration of the polypeptides KVp-N1, KVp-R14 and KVp-C15) were used as examples, human lung cancer cell line A549 cells (purchased from Cell Resource Center of Institute of Basic Medical Sciences Chinese Academy of Medical Sciences) and 293T cells overexpressing ACE2 protein (293T-ACE2⁺) (purchased from Beijing Sino Biological Science and Technology Co., Ltd.) were used as target cells, to simulate and verify the effects that the polypeptides KVp-N1, KVp-R14, KVp-C15 and their composition prevent the binding of the S1 protein of SARS-CoV-2 or its RBD segment to target cells.

### Experiment 3.1: Effects of Polypeptides and their Composition on the Binding Ability of S1 Protein to Human Lung Cancer Cell Line A549 Cells

(1) A549 cells were inoculated into a 6-well plate at a density of 2×10⁵ cells/well, and were cultured overnight in a 37° C. incubator to allow the cells to adhere to the wall;
(2) S1 protein was diluted with PBS to 1 μM S1 protein solution, and then it was co-incubated with 0.1 μM, 1 μM, 10 μM, 100 μM of the polypeptides KVp-N1, KVp-R14, and KVp-C15 (prepared by diluting 10 mM stock solutions of the polypeptides KVp-N1, KVp-R14, KVp-C15 respectively with complete medium) for 1 h, respectively, and the S1 protein solution that was not co-incubated with the polypeptides KVp-N1, KVp-R14, and KVp-C15 was simultaneously set as the control group;

At the same time, S1 protein was diluted with PBS to 1 μM S1 protein solution, and then it was co-incubated with three single polypeptides KVp-N1, KVp-R14, KVp-C15 (the concentration of each single polypeptide was 1 μM, and the stock solution was diluted with complete medium) and the polypeptide composition (the concentration of each polypeptide was 1 μM, and the stock solution was diluted with complete medium) at room temperature for 1 h, and the S1 protein solution that was not co-incubated with the polypeptide was simultaneously set as the control group;
(3) The mixture of S1 protein and polypeptide (KVp-N1, KVp-R14, KVp-C15 and polypeptide composition) of the above groups and the mixture of the control group were added into a cell culture system, respectively, and ensure that the final concentration of S1 protein was 10 nM;
(4) After co-cultivation for 1 h, the cell culture system was washed twice with PBS, the total protein was extracted by lysing cells with RIPA lysate, and the content of S1 protein in the total protein was detected by Western Blot.

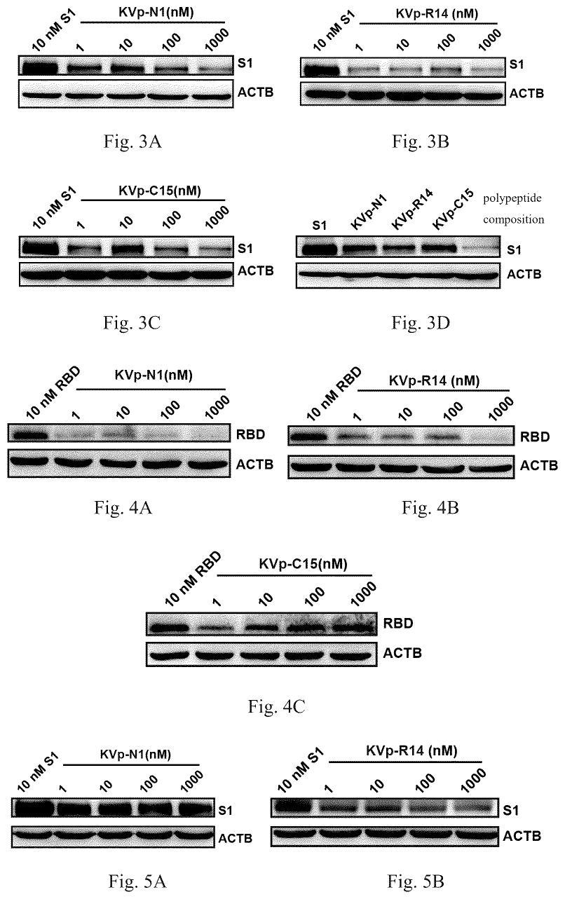

The results of different concentrations of the polypeptides KVp-N1, KVp-R14, and KVp-C15 are shown in FIGS. 3A-3C, respectively. It can be seen that there is a strong positive signal of S1 protein in the control group, indicating that the S1 protein has bound to A549 cells; but when the S1 protein was first incubated with the polypeptides KVp-N1, KVp-R14, and KVp-C15 for 1 h, and then was added to the cell culture system, the intensity of S1 protein signal is significantly reduced, and within the range of polypeptide concentration from 1 nM to 1000 nM, the intensity of the S1 protein positive signal become weaker with the increase of the concentration of the polypeptide KVp-N1, KVp-R14, and KVp-C15, indicating that the polypeptides KVp-N1, KVp-R14, and KVp-C15 inhibit the binding of S1 protein to A549 cells.

The results of the polypeptide composition are shown in FIG. 3D. It can be seen that there is a strong positive signal of S1 protein in the control group, indicating that the S1 protein has bound to A549 cells; but when S1 protein was first incubated with the polypeptide (KVp-N1, KVp-R14, KVp-C15 and the polypeptide composition) for 1 h, and then was added to a cell culture system, the intensity of S1 protein signal is significantly reduced, indicating that the polypeptides inhibit the binding of S1 protein to A549 cells, and the ability of the polypeptide composition inhibiting the binding of S1 protein to A549 cells is significantly stronger than that of the three single polypeptide, showing the joint effect (this effect will be further described in Example 4 below) of the three single polypeptide in the polypeptide composition.

### Experiment 3.2: Effects of Polypeptides and their Composition on the Binding Ability of RBD Protein to Human Lung Cancer Cell Line A549 Cells

The specific operation of Experiment 3.2 was the same as that in Experiment 3.1 above, except that RBD protein solution was co-incubated with 0.1 μM, 1 μM, 10 μM, 100 μM of the polypeptides KVp-N1, KVp-R14, KVp-C15, the polypeptides KVp-N1, KVp-R14, KVp-C15 and polypeptide composition instead of the S1 protein solution.

The content of RBD protein in the total protein was detected by Western Blot. The results of different concentrations of the polypeptides KVp-N1, KVp-R14, and KVp-C15 are shown in FIGS. 4A-4C, respectively. It can be seen that the control group has a strong positive signal of RBD protein, indicating that RBD protein has bound to A549 cells; but after RBD protein was incubated with the polypeptides KVp-N1, KVp-R14, and KVp-C15 respectively, the binding amount of RBD protein on A549 cells is significantly less than that of the control group, indicating that the polypeptides KVp-N1, KVp-R14, and KVp-C15 inhibit the binding of RBD protein to A549 cells, and the inhibitory effect is more obvious with the increase of polypeptide concentration.

Similar to FIG. 3D, it can be seen that the control group had a strong positive signal of RBD protein, indicating that RBD protein has bound to A549 cells; but when RBD protein was first incubated with the polypeptide (KVp-N1, KVp-R14, KVp-C15 and the polypeptide composition) for 1 h, and then was added to a cell culture system, the intensity of RBD protein signal is significantly reduced, indicating that the polypeptides inhibit the binding of RBD protein to A549 cells, and the ability of the polypeptide composition inhibiting the binding of RBD protein to A549 cells is significantly stronger than that of the three single polypeptide.

### Experiment 3.3: Effects of Polypeptides and their Composition on the Binding Ability of RBD Protein to Human Lung Cancer Cell Line 293T-ACE2⁺ Cells

The specific operation of Experiment 3.3 was the same as that in Experiment 3.2 above, except that 293T-ACE2⁺ cells was used as the target cells instead of A549 cells.

The content of RBD protein in the total protein was detected by Western Blot. The results of different concentrations of the polypeptides KVp-N1, KVp-R14, and KVp-C15 are shown in FIGS. 6A-6C, respectively. It can be seen that the control group has a strong positive signal of RBD protein, indicating that RBD protein has bound to 293T-ACE2⁺ cells; but after RBD protein was incubated with the polypeptides KVp-N1, KVp-R14, and KVp-C15 respectively, the binding amount of RBD protein on 293T-ACE2⁺ cells is significantly less than that of the control group, indicating that the polypeptides KVp-N1, KVp-R14, and KVp-C15 inhibit the binding of RBD protein to 293T-ACE2⁺ cells, and the inhibitory effect is more obvious with the increase of the polypeptide concentration.

Based on the above experimental results, it is proved that the polypeptide KVp-N (KVp-N1 and KVp-N1N), the polypeptide KVp-R (KVp-R14 and KVp-R14N), and the polypeptide KVp-C (KVp-C15 and KVp-C15N) can prevent the binding of S1 protein or its RBD protein to the target cells (human lung cancer cell line A549 cells and human lung cancer cell line 293T-ACE2⁺ cells).

The results of the polypeptide composition are shown in FIG. 6D. It can be seen that the control group has a strong positive signal of RBD protein, indicating that RBD protein has bound to 293T-ACE2⁺ cells; but when S1 protein was first incubated with the polypeptide (KVp-N1, KVp-R14, KVp-C15 and the polypeptide composition) for 1 h, and then was added to a cell culture system, the intensity of RDB protein signal is significantly reduced, indicating that the polypeptides inhibit the binding of RBD protein to 293T-ACE2⁺ cells, and the ability of the polypeptide composition inhibiting the binding of RBD protein to 293T-ACE2⁺ cells is significantly stronger than that of the three single polypeptide, showing the joint effect (this effect will be further described in Example 4 below) of the three single polypeptide in the polypeptide composition.

### Experiment 3.4: Effects of Polypeptides on the Binding Ability of S1 Protein to Human Lung Cancer Cell Line 293T-ACE2⁺ Cells

The specific operation of Experiment 3.4 was the same as that in Experiment 3.1 above, except that 293T-ACE2⁺ cells was used as the target cells instead of A549 cells.

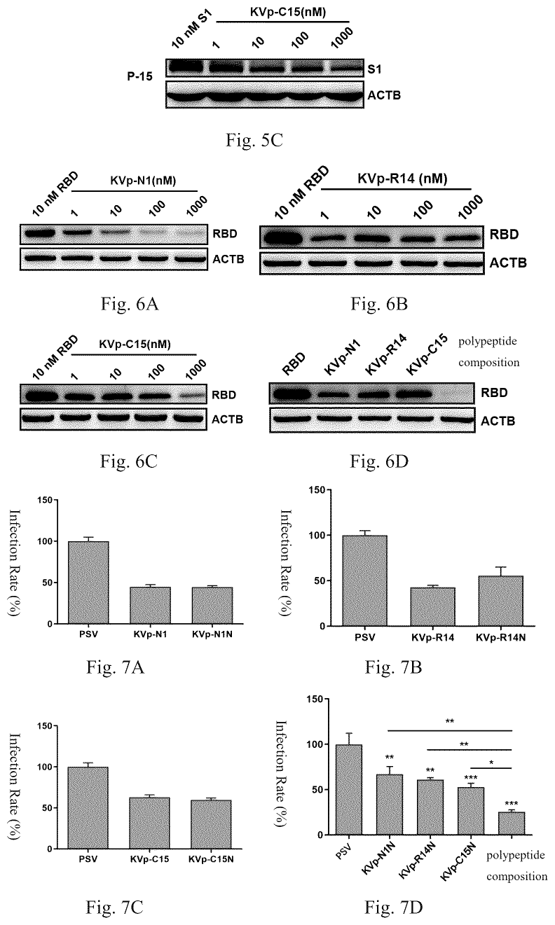

The content of S1 protein in the total protein was detected by Western Blot. The results of different concentrations of polypeptides KVp-N1, KVp-R14, and KVp-C15 are shown in FIGS. 5A-5C, respectively. It can be seen that the control group has a strong positive signal for S1 protein, indicating that S1 protein has bound to 293T-ACE2⁺ cells; but when S1 protein was first incubated with the polypeptides KVp-N1, KVp-R14, and KVp-C15 for 1 h, and then was added to a cell culture system, the intensity of S1 protein signal is significantly reduced, indicating that the polypeptides KVp-N1, KVp-R14, and KVp-C15 inhibit the binding of S1 protein to 293T-ACE2⁺ cells, and the inhibitory effect is more obvious with the increase of the polypeptide concentration.

Similar to FIG. 6D, it can be seen that the control group has a strong positive signal for S1 protein, indicating that S1 protein has bound to 293T-ACE2⁺ cells; but when S1 protein was first incubated with the polypeptide (KVp-N1, KVp-R14, KVp-C15 and the polypeptide composition) for 1 h, and then was added to a cell culture system, the intensity of S1 protein signal is significantly reduced, indicating that the polypeptides inhibit the binding of S1 protein to 293T-ACE2⁺ cells, and the ability of the polypeptide composition inhibiting the binding of S1 protein to A549 cells is significantly stronger than that of the three single polypeptide.

### Example 4: Neutralization Ability of Polypeptide and Composition Thereof to SARS-CoV-2 Pseudovirus

In this example, the pseudovirus neutralization method was used to detect the blocking effect of the polypeptides KVp-N1N, KVp-R14N, KVp-C15N synthesized in Example 1 and their equimolar concentration mixture (i.e. the polypeptide composition composed of equimolar concentrations of the polypeptides KVp-N1, KVp-R14 and KVp-C15) on the infected cells of SARS-CoV-2 pseudovirus, wherein the SARS-CoV-2 pseudovirus (PSV) used was purchased from Jiman Biotechnology (Shanghai) Co., Ltd., which is tagged with luciferase and green fluorescent protein (GFP). Specifically include the following operations:
(1) 293T-ACE2⁺ cells were inoculated into a 96-well plate with 2×10⁴ cells per well, and cultured overnight to make them adhere to the wall;
(2) Pseudoviruses (>10⁶ copies/mL) with luciferase and green fluorescent protein (GFP) tags were mixed and incubated for 1 h respectively with 10 μM of polypeptides KVp-N1 and KVp-N1N (prepared respectively by diluting 10 mM stock solution of the polypeptides KVp-N1 and KVp-N1N with complete medium), polypeptides KVp-R14 and KVp-R14N (prepared respectively by diluting 10 mM stock solution of the polypeptides KVp-R14 and KVp-R14N with complete medium), polypeptides KVp-C15 and KVp-C15N (prepared respectively by diluting 10 mM stock solution of the polypeptides KVp-C15 and KVp-C15N with complete medium), and were mixed and incubated for 1 h respectively with dose gradients of 100 μM, 10 μM, 1 μM, 100 nM, 10 nM, 1 nM of polypeptide KVp-N1N (prepared respectively by diluting 10 mM stock solution of the polypeptide KVp-N1N with complete medium), dose gradients of 100 μM, 10 μM, 1 μM, 100 nM, 10 nM, 1 nM of polypeptide KVp-R14N (prepared respectively by diluting 10 mM stock solution of the polypeptide KVp-R14N with complete medium), dose gradients of 100 PM, 10 μM, 1 μM, 100 nM, 10 nM, 1 nM of polypeptide KVp-C15N (prepared respectively by diluting 10 mM stock solution of the polypeptide KVp-C15N with complete medium), and three single polypeptide (KVp-N1N, KVp-R14N, KVp-C15N, the doses were 10 μM) solutions and polypeptide composition (the concentration of each polypeptide was 10 μM) solutions;
(3) The mixtures of pseudovirus and the polypeptide mixed and incubated were added into the cell culture system in step (1) to infect 293T-ACE2⁺ cells at 37° C. for 1 h, and then switched to normal medium and continued to culture for 47 h. After culturing, the cells were washed once with PBS, and detected chemiluminescence with luciferase detection kits (Promega, Cat. No. E1500), and the infection rate of pseudoviruses in different groups was calculated accordingly.

The results of polypeptides KVp-N1 and KVp-N1N, polypeptides KVp-R14 and KVp-R14N, and polypeptides KVp-C15 and KVp-C15N are shown in FIGS. 7A-7C, wherein PSV represents the infection rate of the SARS-CoV-2 pseudoviruses that was not incubated with the polypeptides. KVp-N1 and KVp-N1N, KVp-R14 and KVp-R14N, KVp-C15 and KVp-C15N represent respectively the infection rate of pseudoviruses to 293T-ACE2⁺ cells after the pseudoviruses and the polypeptides with the concentration of 10 μM was mixed and incubated. It can be seen clearly that at the same dose, polypeptide KVp-N1 and polypeptide KVp-N1N, polypeptide KVp-R14 and polypeptide KVp-R14N, polypeptide KVp-C15 and polypeptide KVp-C15N all have strong and basically the same pseudovirus neutralization ability.

FIG. 7D shows the infection rate of SARS-CoV-2 pseudovirus incubated with single polypeptide KVp-N1N, KVp-R14N, KVp-C15N with the dose of 10 μM, and their mixed polypeptide composition on 293T-ACE2⁺ cells.

According to the results shown in FIG. 7D, it can be seen obviously that the infection rate of SARS-CoV-2 pseudoviruses to 293T-ACE2⁺ cells (the infection rate is only about 25%) reduces significantly after SARS-CoV-2 pseudovirus were co-incubated with the polypeptide composition, which is formed by mixing the three polypeptides (KVp-N1N, KVp-R14N, KVp-C15N), indicating that the reduction of the infection rate does not depend on the increase of the total concentration of the three polypeptides, but is due to the different targeting sites of each polypeptide in the polypeptide composition, which can work together in inhibiting the binding of the S1 protein of SARS-CoV-2 or its RBD protein to the target cells, making it have a significant effect on neutralizing the infectivity of SARS-CoV-2 pseudovirus.

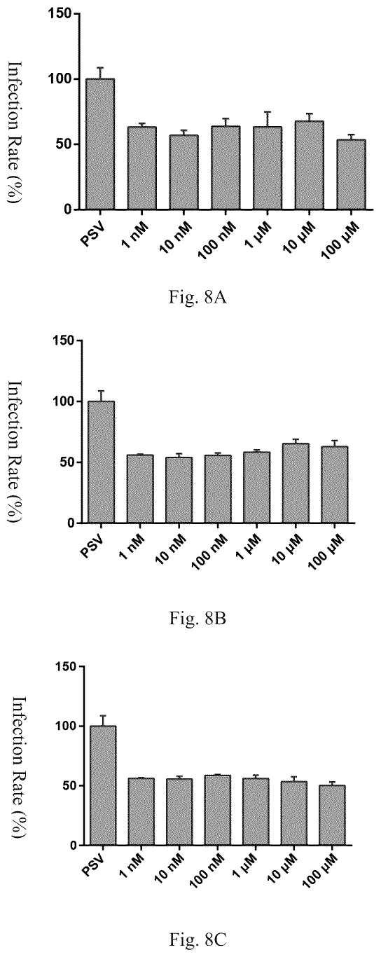

FIGS. 8A-8C respectively show the infection rate of SARS-CoV-2 pseudoviruses, after SARS-CoV-2 pseudoviruses was co-incubated with the polypeptides KVp-N1N, KVp-R14N, and KVp-C15N in the dose range of 1 nM to 100 μM, to 293T-ACE2⁺ cells. FIG. 8A shows the infection rate of pseudoviruses PSV, after pseudoviruses PSV was co-incubated with the polypeptide KVp-N1N in the dose range of 1 nM to 100 μM, to 293T-ACE2⁺ cells; FIG. 8B shows the infection rate of pseudoviruses PSV, after pseudoviruses PSV was co-incubated with the polypeptide KVp-R14N in the dose range of 1 nM to 100 μM, to 293T-ACE2⁺ cells; FIG. 8C shows the infection rate of pseudoviruses PSV, after pseudoviruses PSV was co-incubated with the polypeptide KVp-C15N in the dose range of 1 nM to 100 μM, to 293T-ACE2⁺ cells.

According to the results shown in FIG. 8A-8C, it is proved that the polypeptides KVp-N (KVp-N1 and KVp-N1N), KVp-R (KVp-R14 and KVp-R14N), KVp-C (KVp-C15 and KVp-C15N) all have strong neutralization ability to SARS-CoV-2 pseudovirus, and the neutralization ability is about 50%. And in the tested dose range of 1 nM to 100 μM, there is no dependence between the efficiency of the polypeptides inhibiting the infection of pseudovirus to cells and their concentrations, showing that the efficiency of inhibiting the infection of SARS-CoV-2 pseudovirus to 293T-ACE2⁺ cells is basically the same.

### Example 5: Cytotoxicity of Polypeptide and Composition Thereof

In this example, the cytotoxicity of the polypeptides KVp-N1, KVp-N1N, KVp-R14, KVp-R14N, KVp-C15 and KVp-C15N prepared in Example 1 is detected by using 293T-ACE2⁺ cells, and the specific operations included the following operations.
(1) 293T-ACE2⁺ cells grown in logarithmic phase were taken, and the density of the cells was adjusted to 10′ cells/mL with complete medium, and the cells were inoculated into 96-well plate, 100 μL per well;
(2) The polypeptides KVp-N1, KVp-N1N, KVp-R14, KVp-R14N, KVp-C15 and KVp-C15N were taken and added to the cell culture system of each well respectively, so that the final concentrations of the polypeptides were 1 nM, 10 nM, 100 nM, 1 μM, 10 μM, 100 μM, respectively, and were cultured at 37° C. for 48 h;
(3) The cells were washed once with PBS after discarding supernatant after culturing, 120 μL of cell counting kit-8 (CCK-8) working solution (CCK-8 working solution is obtained by mixing CCK8 stock solution and complete medium in a volume ratio of 1:10, CCK8 stock solution was purchased from Dojindo Molecular Technologies) was added to each well. After mixing, the system was incubated at 37° C. for 3 h, then the absorbance values at 450 nm and 630 nm (reference wavelength) were detected to calculate the cell viability.

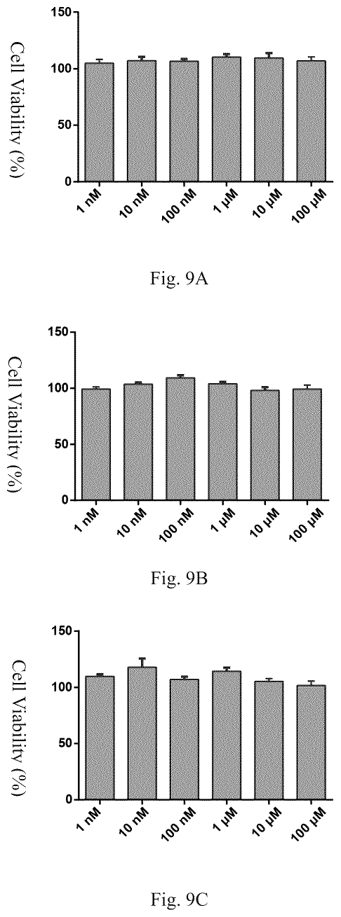

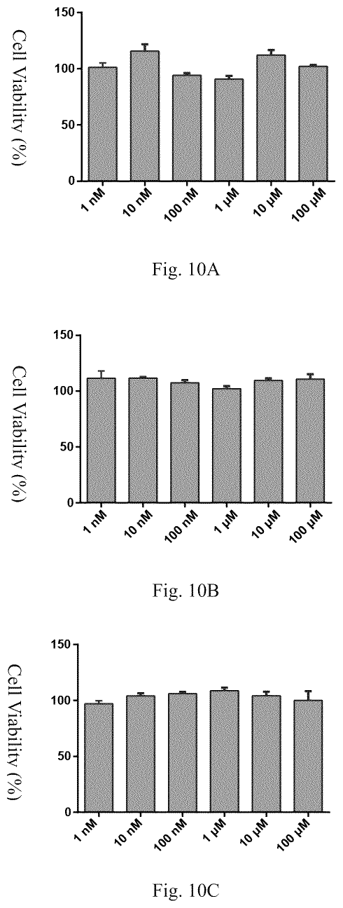

The results are shown in FIGS. 9A-9C and FIGS. 10A-10C, wherein FIG. 9A shows the effect of the polypeptide KVp-N1 in the tested dose range on 293T-ACE2⁺ cells viability; FIG. 10A shows the effect of the polypeptide KVp-N1N in the tested dose range on 293T-ACE2⁺ cells viability; FIG. 9B shows the effect of the polypeptide KVp-R14 in the tested dose range on 293T-ACE2⁺ cells viability; FIG. 10B shows the effect of the polypeptide KVp-R14N in the tested dose range on 293T-ACE2⁺ cells viability; FIG. 9C shows the effect of the polypeptide KVp-C15 in the tested dose range on 293T-ACE2⁺ cells viability; FIG. 10C shows the effect of the polypeptide KVp-C15 in the tested dose range on 293T-ACE2⁺ cells viability. It can be seen that the effects of the six polypeptides on 293T-ACE2⁺ cells viability in the tested dose range are not significant; combined with the results shown in FIGS. 7A-7C and 8A-8C, in the tested dose range of 1 nM to 100 μM, a single polypeptide has no cytotoxic effect on 293T-ACE2⁺ cells, and has good SARS-CoV-2 pseudovirus neutralization ability; then the polypeptide composition mixed with any two or more polypeptides of KVp-N1, KVp-N1N, KVp-R14, KVp-R14N, KVp-C15 and KVp-C15N in the tested dose range of 1 nM to 100 μM must also have no cytotoxic effect on 293T-ACE2⁺ cells.

In summary of the results of the above examples, it can be seen that the polypeptides and compositions thereof (for example, mixed with any two or more polypeptides of KVp-N1, KVp-R14, KVp-C15, KVp-N1N, KVp-R14N and KVp-C15N) provided in the invention can bind to S1 protein of SARS-CoV-2 virus and its RBD segment, and thereby block the binding of S1 protein of SARS-CoV-2 virus and its RBD segment to its targets (such as angiotensin-converting enzyme 2 (ACE2) on the surface of ciliated bronchial epithelial cells and type II lung cells), and thus can prevent SARS-CoV-2 virus from infecting epithelial cells and lung cells, and exhibit a strong neutralization ability to SARS-CoV-2 pseudoviruses, and all kinds of the polypeptides in the polypeptide composition work together, showing a significantly higher neutralization ability to SARS-CoV-2 pseudoviruses than a single polypeptide used.

The infection mechanism of Coronavirus disease 2019 is that the SARS-CoV-2 virus binds to angiotensin-converting enzyme 2 (ACE2) of ciliated bronchial epithelial cells and type II lung cells through the RBD domain of its surface spike protein S1 subunit, which causes cells to be infected. therefore, the polypeptides and compositions thereof provided by the invention can not only be used to prepare blockers that block the binding of S1 protein of the SARS-CoV-2 virus and its RBD segment to ACE2, but also can be used to prepare medicine for treating Coronavirus disease 2019, to achieve the purpose of targeted treatment of Coronavirus disease 2019.

The polypeptides and polypeptide compositions provided by the invention have the characteristics of low molecular weight, high biological activity, and hard to accumulate in the body. They can not only be used as drugs themselves, but can also be used in combination with other drug molecules (such as anti-inflammatory drugs, anti-infective drugs, etc.), and there is less interaction with other drugs when combinating; at the same time, these polypeptides and their compositions also have the advantages of easy synthesis and molecular modification, and low production costs, so they can be produced and applied on a large scale.

### INDUSTRIAL APPLICABILITY

The polypeptide and polypeptide composition provided by the invention can block the binding of SARS-CoV-2 virus to a key target protein angiotensin-converting enzyme (ACE2) protein on the surface of a host cell thereof, thereby providing a feasible targeting drug for the treatment of Coronavirus disease 2019, and is suitable for industrial application.

1. A polypeptide composition specifically binding to SARS-CoV-2 spike protein, wherein the polypeptide composition comprises one or more of polypeptide KVp-N, polypeptide KVp-R and polypeptide KVp-C; the polypeptide KVp-N contains the amino acid sequence shown in SEQ ID NO:1, the polypeptide KVp-R contains the amino acid sequence shown in SEQ ID NO:3, and the polypeptide KVp-C contains the amino acid sequence shown in SEQ ID NO:5.

2. The polypeptide composition according to claim 1, wherein the ends of the polypeptide KVp-N, the polypeptide KVp-R and the polypeptide KVp-C are modified with a sulfhydryl-containing peptide fragment; preferably, the amino acid sequence of the sulfhydryl-containing peptide fragment is CCPPPP.

3. The polypeptide composition according to claim 1, wherein
the amino acid sequence of the polypeptide KVp-N modified with a sulfhydryl-containing peptide fragment is shown in SEQ ID NO:2;
The amino acid sequence of the polypeptide KVp-R modified with a sulfhydryl-containing peptide fragment is shown in SEQ ID NO:4;
The amino acid sequence of the polypeptide KVp-C modified with a sulfhydryl-containing peptide fragment is shown in SEQ ID NO:6.

4. The polypeptide composition according to claim 1, wherein each polypeptide is mixed in any proportion; preferably, the polypeptide KVp-N, the polypeptide KVp-R and the polypeptide KVp-C are mixed in an equimolar ratio.

5.-11. (canceled)

12. Use of the polypeptide composition of claim 1 in the preparation of a medicine for treating Coronavirus disease 2019, preferably, the coronavirus is SARS-CoV-2.

13. Use of the polypeptide composition of claim 1 in the preparation of an blocking agent for blocking the binding of SARS-CoV-2 surface spike S1 protein to angiotensin-converting enzyme 2 (ACE2); preferably, the angiotensin-converting enzyme 2 (ACE2) exists on the surface of ciliated bronchial epithelial cells or type II lung cells.

14. The use according to claim 12, wherein the polypeptide composition or the polypeptide is used to specifically block the binding of SARS-CoV-2 surface spike S1 protein to angiotensin-converting enzyme 2 (ACE2).

15. The use according to claim 14, wherein the polypeptide composition or the polypeptide is used to specifically bind to the SARS-CoV-2 surface spike S1 protein to block the binding of the SARS-CoV-2 surface spike S1 protein to angiotensin-converting enzyme 2 (ACE2).

16. The use according to claim 14, wherein the polypeptide composition or the polypeptide is used to specifically bind to the RBD domain of the SARS-CoV-2 surface spike S1 protein subunit to block the binding of the SARS-CoV-2 surface spike S1 protein to angiotensin-converting enzyme 2 (ACE2).

17. The use according to claim 13, wherein the polypeptide composition or the polypeptide is used to specifically block the binding of SARS-CoV-2 surface spike S1 protein to angiotensin-converting enzyme 2 (ACE2).

18. The use according to claim 17, wherein the polypeptide composition or the polypeptide is used to specifically bind to the SARS-CoV-2 surface spike S1 protein to block the binding of the SARS-CoV-2 surface spike S1 protein to angiotensin-converting enzyme 2 (ACE2).

19. The use according to claim 17, wherein the polypeptide composition or the polypeptide is used to specifically bind to the RBD domain of the SARS-CoV-2 surface spike S1 protein subunit to block the binding of the SARS-CoV-2 surface spike S1 protein to angiotensin-converting enzyme 2 (ACE2).


SEQUENCE LISTING (7 sequences)

SEQ ID NO: 1 - protein, 21 aa, Artificial Sequence
  polypeptide
GDGVYYPRDVFDSSVLDSTQR

SEQ ID NO: 2 - protein, 27 aa, Artificial Sequence
  polypeptide
CCPPPPGDGVYYPRDVFDSSVLDSTQR

SEQ ID NO: 3 - protein, 20 aa, Artificial Sequence
  polypeptide
GDLFDDSNLDPFRDRISTRR

SEQ ID NO: 4 - protein, 26 aa, Artificial Sequence
  polypeptide
CCPPPPGDLFDDSNLDPFRDRISTRR

SEQ ID NO: 5 - protein, 21 aa, Artificial Sequence
  polypeptide
GDGVYYPRDVFDSSVLDSTQR

SEQ ID NO: 6 - protein, 27 aa, Artificial Sequence
  polypeptide
CCPPPPGDGVYYPRDVFDSSVLDSTQR

SEQ ID NO: 7 - protein, 6 aa, Artificial Sequence
  polypeptide
CCPPPP